(19) United States
(12) Patent Application Publication
(10) Pub. No.: US 2017/0270331 A1
(43) Pub. Date: Sep. 21, 2017

(54) SCANNER GLOVES AND REAL-TIME TRACKING SYSTEM

(71) Applicant: Tobias Hoeink, Houston, TX (US)

(72) Inventor: Tobias Hoeink, Houston, TX (US)

(73) Assignee: Baker Hughes Incorporated, Houston, TX (US)

(21) Appl. No.: 15/073,297

(22) Filed: Mar. 17, 2016

(51) Int. Cl. G06K 7/14; G06Q 10/08; A61B 5/00; A41D 19/00; A61B 5/024

(52) U.S. Cl. CPC G06K 7/1413; A41D 19/0024; A41D 19/0044; A61B 5/02438; A61B 5/6806; G06Q 10/087; G06K 2007/10524

(57) ABSTRACT

A scanning and monitoring glove system includes a glove member having a palm portion, a thumb portion and a plurality of finger portions, a product scanner integrated into the glove member, a digital camera integrated into one of the plurality of finger portions of the glove member, and a biometric scanning device integrated into the glove member. The biometric scanning device being operable to sense biometric data associated with a wearer of the glove member.

### BACKGROUND

In complex operations, tracking numerous products, materials, tools, may be a herculean task. Accurately tracking materials, products and tools during various production operations and/or in various production facilities improves an overall profitability. That is, proper and accurate inventory control allows for better planning, reduced warehousing costs, more accurate accounting and increased profitability.

### SUMMARY

A scanning and monitoring glove system includes a glove member having a palm portion, a thumb portion and a plurality of finger portions, a product scanner integrated into the glove member, a digital camera integrated into one of the plurality of finger portions of the glove member, and a biometric scanning device integrated into the glove member. The biometric scanning device being operable to sense biometric data associated with a wearer of the glove member.

An inventory tracking and worker monitoring system including a computer system having a central processing unit (CPU), a non-volatile memory and an input device operatively connected to the CPU, and a scanning and monitoring glove system including a glove member having a palm portion, a thumb portion and a plurality of finger portions, a product scanner integrated into the glove member, a digital camera integrated into one of the plurality of finger portions of the glove member, and a biometric scanning device integrated into the glove member, the biometric scanning device being operable to sense biometric data associated with a wearer of the glove member.

### BRIEF DESCRIPTION OF THE DRAWINGS

Referring now to the drawings wherein like elements are numbered alike in the several Figures:

FIG. 1 depicts an individual wearing a scanning and monitoring glove system in accordance with an aspect of an exemplary embodiment;

FIG. 2 depicts the scanning and monitoring glove system of FIG. 1; and

FIG. 3 depicts the scanning and monitoring glove system interfacing with an inventory control and worker monitoring system in accordance with an aspect of an exemplary embodiment.

### DETAILED DESCRIPTION

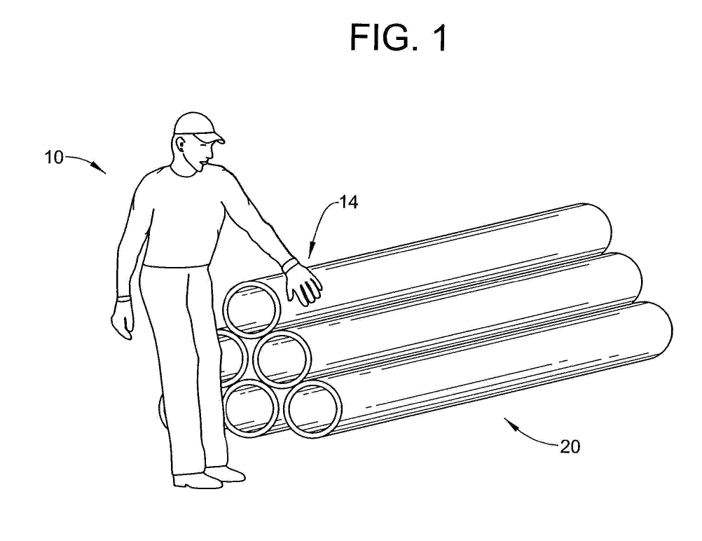

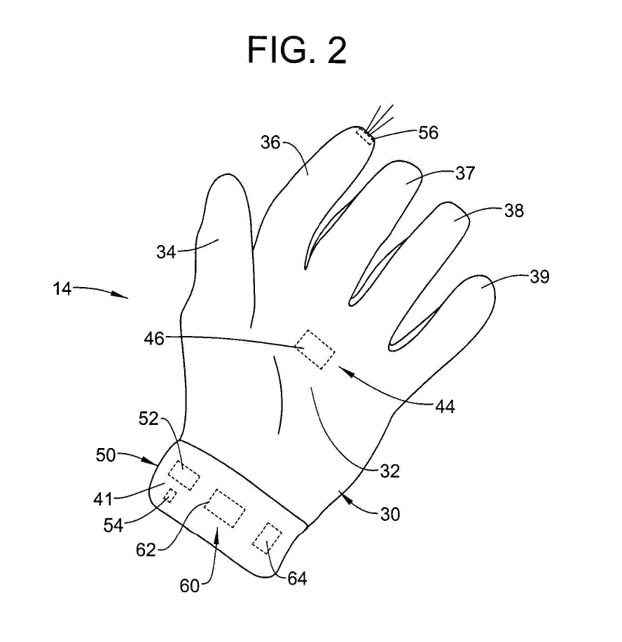

An individual 10 wearing a scanning and monitoring glove system 14 in accordance with an exemplary embodiment is illustrated in FIG. 1. Individual 10 may employ scanning and monitoring glove system 14 to track inventory, such as downhole tubulars 20, tools (not shown) and/or other materials. As seen in FIG. 2, scanning and monitoring glove system 14 includes a glove member 30 having a palm portion 32, a thumb portion 34, a plurality of finger portions 36-39, and a cuff portion 41.

In accordance with an aspect of an exemplary embodiment, glove member 30 includes a product scanner 44 that may be employed to detect materials, tools, supplies or other objects associated with, for example, a production operation. For example, product scanner 44 may be employed to scan tubulars 20 prior to introduction into a well bore. In this manner, a more accurate count of tubulars may be maintained. Product scanner 44 may take the form of a radio frequency identification (RFID) scanner 46. Alternatively, product scanner 44 may take the form of a bar code scanner.

Glove member 30 may also include an output system 50 that provides an interface with other systems during and/or after individual 10 completes a shift. Output system 50 may include a wireless transmitter 52 and/or a communication portion 54 that facilitates a wired interface with glove member 30. In this manner, individual 10 may download inventory data as well as other data either periodically during a shift or at the end of a workday as will be detailed more fully below.

In further accordance with an exemplary embodiment, glove member 30 includes a digital camera 56 integrated into finger portion 36. Digital camera 56 may be employed by individual 10 to view areas that may be inaccessible, and/or areas that are not readily viewable to the human eye. Digital camera 56 is operatively connected to wireless transmitter 52 and/or communication portion 54 to allow transmission and/or downloading of captured images.

In still further accordance with an aspect of an exemplary embodiment, glove member 30 may include a biometric scanning device 60. Biometric scanning device 60 may take the form of a heart rate monitor 62 incorporated into cuff portion 41. Biometric scanning device 60 may take on other forms such as temperature sensing devices, hydration sensing devices, heart beat/pulse sensing, blood pressure sensing, and/or combinations thereof. Biometric scanning device 60 is operatively connected to, for example, wireless transmitter 52 thereby allowing a supervisor to determine various biometric statistics associated with individual 10. In yet still further accordance with an exemplary aspect, glove member 30 may further include a global positioning system (GPS) device 64. GPS device 64 may allow a supervisor other personnel to track individual 10. In hazardous work environments, tracking health and location of employees may improve overall work place safety and productivity.

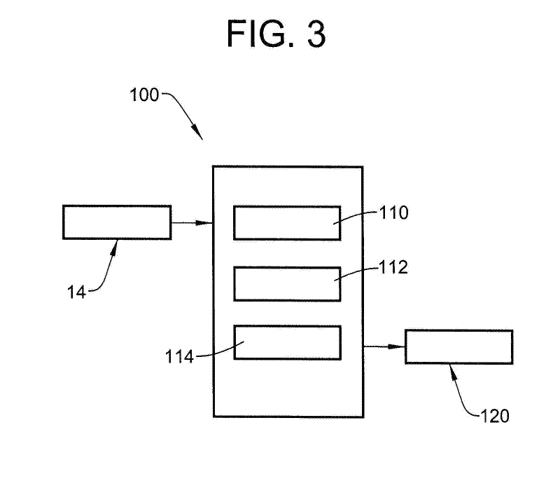

Scanning and monitoring glove system 14 may be integratable into an inventory tracking and working monitoring system 100 such as shown in FIG. 3. Inventory tracking and worker tracking system 100 includes a central processing unit (CPU) 110 operably connected with a non-volatile memory 112 and an input device 114. Input device 114 receives data from glove member 30 transmitted through wireless transmitter 52 or passed through a connector cable (not separately labeled) interfacing with communication portion 54. Collected data may be output through an output device 120 which could take the form of a display, a smart phone or the like.

Inventory tracking and working monitoring system 100 may track inventory used, location of inventory, and or worker wellbeing. In the event that additional inventory is desired, inventory tracking and working monitoring system 100 may automatically signal a warehouse (not shown) to request a delivery. Additionally, inventory tracking and working monitoring system 100 may provide alerts to supervisory personnel indicating that a particular worker may be in distress. Often times, individual 10 may be in an operating environment that creates physical stresses. High temperature, low temperature, and arid environments all place physical stresses on workers. If a particular worker is approaching a predetermined stress limit, inventory tracking and working monitoring system 100 may signal a need for intervention. Intervention may take the form of fluids, or the need for medical personnel.

Embodiment 1: A scanning and monitoring glove system comprising: a glove member including a palm portion, a thumb portion and a plurality of finger portions; a product scanner integrated into the glove member; a digital camera integrated into one of the plurality of finger portions of the glove member; and a biometric scanning device integrated into the glove member, the biometric scanning device being operable to sense biometric data associated with a wearer of the glove member.

Embodiment 2: The scanning and monitoring glove system according to embodiment 1, further comprising: a memory device incorporated into the glove member and operatively connected to one or more of the product scanner, the digital camera, and the biometric scanning device.

Embodiment 3: The scanning and monitoring glove system according to embodiment 2, further comparing: a connection port integrated into the glove member and operatively connected to the memory, the connection port being connectable to a computer system through a wired connection.

Embodiment 4: The scanning and monitoring glove system according to embodiment 1, further comprising: a wireless communication device configured to wirelessly transmit data between the glove member and a computer system.

Embodiment 5: The scanning and monitoring glove system according to embodiment 1, further comprising: a global positioning system (GPS) device operable to determine a geographic position of the wearer.

Embodiment 6: The scanning and monitoring glove system according to embodiment 1, wherein the product scanner comprises a radio frequency identification (RFID) reader.

Embodiment 7: The scanning and monitoring glove system according to embodiment 1, wherein the product scanner comprises a bar code reader.

Embodiment 8: The scanning and monitoring glove system according to embodiment 1, wherein the biometric scanning device comprises a heart rate monitor.

Embodiment 9: The scanning and monitoring glove system according to embodiment 1, wherein the product scanner is integrated into the palm portion.

Embodiment 10: The scanning and monitoring glove system according to embodiment 1, wherein the glove member includes a cuff portion, the biometric scanning device being integrated into the cuff portion.

Embodiment 11: An inventory tracking and worker monitoring system comprising: a computer system including a central processing unit (CPU), a non-volatile memory and an input device operatively connected to the CPU; and a scanning and monitoring glove system comprising: a glove member including a palm portion, a thumb portion and a plurality of finger portions; a product scanner integrated into the glove member; a digital camera integrated into one of the plurality of finger portions of the glove member; and a biometric scanning device integrated into the glove member, the biometric scanning device being operable to sense biometric data associated with a wearer of the glove member.

Embodiment 12: The inventory tracking and worker monitoring system according to embodiment 11, further comprising: a memory device incorporated into the glove member and operatively connected to one or more of the product scanner, the digital camera, and the biometric scanning device.

Embodiment 13: The inventory tracking and worker monitoring system according to embodiment 12, further comparing: a connection port integrated into the glove member and operatively connected to the memory, the connection port being connectable to a computer system through a wired connection.

Embodiment 14: The inventory tracking and worker monitoring system according to embodiment 11, further comprising: a wireless communication device configured to wirelessly transmit data between the glove member and a computer system.

Embodiment 15: The inventory tracking and worker monitoring system according to embodiment 11, further comprising: a global positioning system (GPS) device operable to determine a geographic position of the wearer.

Embodiment 16: The inventory tracking and worker monitoring system according to Embodiment 11, wherein the product scanner comprises a radio frequency identification (RFID) reader.

Embodiment 17: The inventory tracking and worker monitoring system according to embodiment 11, wherein the product scanner comprises a bar code reader.

Embodiment 18: The inventory tracking and worker monitoring system according to embodiment 11, wherein the biometric scanning device comprises a heart rate monitor.

Embodiment 19: The inventory tracking and worker monitoring system according to embodiment 11, wherein the product scanner is integrated into the palm portion.

Embodiment 20: The inventory tracking and worker monitoring system according to embodiment 11, wherein the glove member includes a cuff portion, the biometric scanning device being integrated into the cuff portion.

While one or more embodiments have been shown and described, modifications and substitutions may be made thereto without departing from the spirit and scope of the invention. Further, while shown and described in connection with downhole operations, the scanning and monitoring glove system may be employed in a wide range of operating environments where it is desirable to track inventory and or worker wellbeing. Accordingly, it is to be understood that the present invention has been described by way of illustrations and not limitation.

1. A scanning and monitoring glove system comprising:
a glove member including a palm portion, a thumb portion and a plurality of finger portions;
a product scanner integrated into the glove member;
a digital camera integrated into one of the plurality of finger portions of the glove member; and
a biometric scanning device integrated into the glove member, the biometric scanning device being operable to sense wellbeing of a wearer of the glove member; and
an output device operably associated with the glove member operable to output inventory signals from the glove member and deliver worker wellbeing alerts.

2. The scanning and monitoring glove system according to claim 1, further comprising: a memory device incorporated into the glove member and operatively connected to one or more of the product scanner, the digital camera, and the biometric scanning device.

3. The scanning and monitoring glove system according to claim 2, further comparing: a connection port integrated into the glove member and operatively connected to the memory, the connection port being connectable to a computer system through a wired connection.

4. The scanning and monitoring glove system according to claim 1, further comprising: a wireless communication device configured to wirelessly transmit data between the glove member and a computer system.

5. The scanning and monitoring glove system according to claim 1, further comprising: a global positioning system (GPS) device operable to determine a geographic position of the wearer.

6. The scanning and monitoring glove system according to claim 1, wherein the product scanner comprises a radio frequency identification (RFID) reader.

7. The scanning and monitoring glove system according to claim 1, wherein the product scanner comprises a bar code reader.

8. The scanning and monitoring glove system according to claim 1, wherein the biometric scanning device comprises a heart rate monitor.

9. The scanning and monitoring glove system according to claim 1, wherein the product scanner is integrated into the palm portion.

10. The scanning and monitoring glove system according to claim 1, wherein the glove member includes a cuff portion, the biometric scanning device being integrated into the cuff portion.

11. An inventory tracking and worker monitoring system comprising:
a computer system including a central processing unit (CPU), a non-volatile memory and an input device operatively connected to the CPU; and
a scanning and monitoring glove system comprising:
a glove member including a palm portion, a thumb portion and a plurality of finger portions;
a product scanner integrated into the glove member;
a digital camera integrated into one of the plurality of finger portions of the glove member;
a biometric scanning device integrated into the glove member, the biometric scanning device being operable to sense wellbeing of a wearer of the glove member; and
an output device operably associated with the glove member operable to output inventory signals from the glove member and deliver worker wellbeing alerts.

12. The inventory tracking and worker monitoring system according to claim 11, further comprising: a memory device incorporated into the glove member and operatively connected to one or more of the product scanner, the digital camera, and the biometric scanning device.

13. The inventory tracking and worker monitoring system according to claim 12, further comparing: a connection port integrated into the glove member and operatively connected to the memory, the connection port being connectable to a computer system through a wired connection.

14. The inventory tracking and worker monitoring system according to claim 11, further comprising: a wireless communication device configured to wirelessly transmit data between the glove member and a computer system.

15. The inventory tracking and worker monitoring system according to claim 11, further comprising: a global positioning system (GPS) device operable to determine a geographic position of the wearer.

16. The inventory tracking and worker monitoring system according to claim 11, wherein the product scanner comprises a radio frequency identification (RFID) reader.

17. The inventory tracking and worker monitoring system according to claim 11, wherein the product scanner comprises a bar code reader.

18. The inventory tracking and worker monitoring system according to claim 11, wherein the biometric scanning device comprises a heart rate monitor.

19. The inventory tracking and worker monitoring system according to claim 11, wherein the product scanner is integrated into the palm portion.

20. The inventory tracking and worker monitoring system according to claim 11, wherein the glove member includes a cuff portion, the biometric scanning device being integrated into the cuff portion.
